(19) United States
(12) Patent Application Publication
(10) Pub. No.: US 2010/0178678 A1
(43) Pub. Date: Jul. 15, 2010

(54) Method for producing cyclopent-4-ene-1,3-diol or cyclopent-4-ene-1,3-diol derivatives

(76) Inventors: Joerg Jung, Floersheim (DE); Bernd Wilhelm Lehnemann, Cologne (DE)

Correspondence Address: PROPAT, L.L.C., 425-C SOUTH SHARON AMITY ROAD, CHARLOTTE, NC, 28211-2841, US

(21) Appl. No.: 12/663,763

(22) PCT Filed: Jun. 5, 2008
(86) PCT No.: PCT/EP08/04482

§ 371 Date: Dec. 9, 2009

(30) Foreign Application Priority Data
Jun. 13, 2007 (DE) 10 2007 027 189.3

(51) Int. Cl. C12P 7/62; C07C 35/06; C07C 69/76; C07C 69/34; C12P 7/02

(52) U.S. Cl. 435/135; 568/838; 560/84; 560/193; 435/155

(57) ABSTRACT

The invention relates to a method for producing cis-cyclopent-4-ene-1,3-diol and cis-cyclopent-4-ene-1,3-dialkanoates by selective cis-1,2-reduction of 4-hydroxycyclopent-2-enone by means of a hydroboron, in the presence of substoichiometric quantities of a trivalent rare earth metal compound, to form cyclopent-4-ene-1,3-diol which can then be optionally reacted with an acylation agent to form cis-cyclopent-4-ene-1,3-dialkanoates, in order to simplify the reprocessing and without intermediate isolation.

Optically active cyclopentene-1,4-diol derivatives are starting materials with a variety of uses for the synthesis of prostaglandins, carbocyclic nucleosides and other biologically active products, which are obtainable in enantiomerically enriched form by enzymatic partial hydrolysis of esters of cis-cyclopent-4-ene-1,3-diol or by enzymatic partial esterification of cis-cyclopent-4-ene-1,3-diol (see, for example, EP 1 428 888 A1 or Tetrahedron Letters 1984, 25 (51), 5875-78). However, a feature common to all these processes is that the starting material used is either cis-cyclopent-4-ene-1,3-diol or a suitable ester (usually the diacetate).

For these precursors, different preparation routes are known, but all are afflicted with disadvantages which complicate the scaleup thereof to the industrial scale, as described below.

A known preparation route consists in the epoxidation of cyclopentadiene by means of peracids or peroxides, followed by copper(I)-catalyzed rearrangement (Marino et al., Tetrahedron Lett. 1983, 24, 5, 441-444) or followed by a palladium(0)-catalyzed rearrangement (Organic Synthesis 1993, Coll. Vol. 8, page 13). However, the handling of organic peroxides on the industrial scale constitutes an unacceptable safety risk and is therefore not an option. The same applies to the handling of cyclopentadiene, which begins to dimerize in a strongly exothermic reaction even at temperatures significantly below room temperature and can therefore be stored and handled only well below room temperature and in very dilute form. Particular problems for industrial implementation arise from the combination of both safety risks, which virtually rules out the application of the process in industry.

A further variant consists in the reaction of cyclopentadiene with photochemically obtained singlet oxygen and reduction of the adduct formed with thiourea (Johnson et al., J. Am. Chem. Soc. 1986, 108, 18, 5655-5656). Likewise for technical reasons, this variant is not very suitable for preparing relatively large amounts of product.

A further variant proceeds from inexpensive furfuryl alcohol, obtained from renewable raw materials, which rearranges at slightly acidic pH to 4-hydroxycyclopent-2-enone. This can then—optionally purified and with a protected hydroxyl function—be converted by selective reduction of the enone to cis-cyclopent-4-ene-1,3-diol or derivatives thereof (Curran et al., Tetrahedron 1997, 53, 6, 1983-2004). This reduction frequently proceeds with insufficient selectivity, such that the product is contaminated with relatively large amounts of undesired trans compounds and saturated cyclopentane derivatives (i.e. products of 1,4 reduction), which are removable by industrial means only with difficulty, if at all. An additional disadvantage is that the starting material of the reduction is obtained initially only with 40 to 60% purity and, owing to its thermal sensitivity, can be purified by distillation only with difficulty. A suitable reduction method would therefore have to be capable of directly reducing the crude product with high selectivity.

The best results are achieved by reduction with alumino- and borohydrides. Aluminohydrides, however, have the disadvantage that the hydroxyl function of the 4-hydroxycyclopent-2-enone starting material has to be protected before the reduction and deprotected again after the reduction, for which the alkanoate group required for the enzymatic hydrolysis is generally unsuitable, since it is likewise reduced by the aluminohydride. This results in a long sequence of reaction steps (rearrangement of furfurol-protection-reduction-deprotection-acylation-enzymatic hydrolysis) with high costs and low overall yields.

In the case of use of borohydrides, only the Luche reduction (Curran et al., Tetrahedron 1997, 53, 6, 1983-2004) achieves satisfactory selectivities, which has the serious disadvantage of working in the presence of a relatively large excess (1 to 2 equivalents) of cerium(III) salts (generally in the form of the hydrates thereof). For instance, for the reduction of 10 g of 4-hydroxycyclopent-2-enone under conventional “Luche conditions”, 38 to 76 g of cerium(III) chloride heptahydrate are required. Apart from the high resulting costs for raw materials and waste disposal, the removal of the salts in the course of the reaction workup is of course also very difficult given these quantitative ratios, especially since the cyclopent-4-ene-1,3-diol reaction product is very hydrophilic, which makes a simple aqueous workup with removal of the salts in the water phase impossible.

Therefore, none of the known processes is suitable for preparing cyclopent-4-ene-1,3-diol or derivatives, for example esters, on the commercial industrial scale at acceptable costs.

It was an object of the present invention to provide a process which is suitable for preparing cyclopent-4-ene-1,3-diol or derivatives, such as organic diesters, from 4-hydroxycyclopent-2-enone, which is obtainable inexpensively by rearrangement of furfurol, wherein
no protecting group operations are required,
no large amounts of cerium(III) salts are required,
no large amounts of heavy metal wastes are obtained,
few process operations are required,
unpurified starting material can be used and
good yields are achieved with high selectivity for the unsaturated cis product.

The present invention achieves this object and relates to a process for preparing cis-cyclopent-4-ene-1,3-diol and cis-cyclopent-4-ene-1,3-dialkanoates by selective cis 1,2 reduction of 4-hydroxycyclopent-2-enone (I) by means of a borohydride (II) in the presence of substoichiometric amounts of a trivalent rare earth metal compound (III) to give cyclopent-4-ene-1,3-diol (IV), which can then optionally, to simplify the workup and with or preferably without intermediate isolation, be reacted with an acylating agent (V) to give cis-cyclopent-4-ene-1,3-dialkanoates (VI). In this process, it is possible to use unpurified crude starting material, and to perform all operations in a one-pot process:

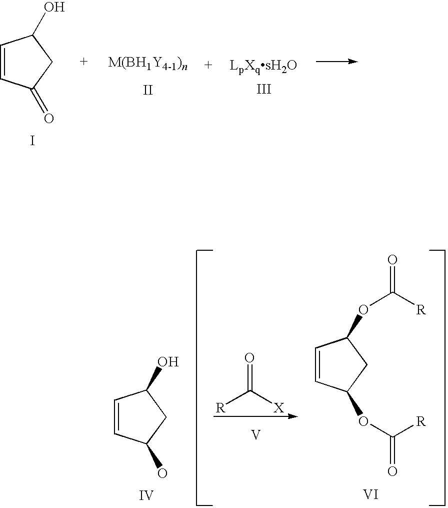

where
M is an alkali metal, an alkaline earth metal, zinc or zirconium,
i is an integer from 1 to 4,
n is an integer from 1 to 4 corresponding to the valency of M,
Y represents monovalent anions which are typically used for the modification of borohydrides, for example cyanide or acetate,
L is any trivalent rare earth metal (or mixtures of trivalent rare earth metals),
X is any anion of organic or inorganic acids,
p and q are each independently integers from 1 to 4, according to the valency of X and the rare earth metal,
s is any number from 0 to 20, which represents the water content of the rare earth metal salt used (the water contents of many rare earth metal salts are nonstoichiometric).

The acylating agent V describes a system composed of a compound for transferring the acyl group R—CO, optionally comprising a base added to scavenge any acid which forms, and optionally comprising an acylation catalyst.

R is an aromatic or aliphatic radical.

According to the invention, an aromatic radical is understood to mean a cyclic molecule with at least one ring in which all atoms are sp²-hybridized and which preferably has (4n+2)π electrons. These aromatic radicals are more preferably C₅-C₁₀-aryls in which one or two carbon atoms may be replaced by heteroatoms such as N, S or O. Examples of such radicals are: phenyl, naphthyl, pyridyl, quinolyl, pyrimidyl, quinazolyl, furyl, benzofuryl, pyrrolyl, indolyl, thiophenyl, benzothiophenyl, imidazolyl, benzimidazolyl, pyrazolyl, indazolyl, oxazolyl, benzoxazolyl, isoxazolyl, benzisoxazolyl, thiazolyl, benzothiazolyl. Preference is given to phenyl, naphthyl, pyridyl, furyl and thiophenyl.

According to the invention, an aliphatic radical is understood to mean a C₁-C₁₈, preferably C₁-C₈ and more preferably C₁-C₄-alkyl radical, which, in the case of >C₂, may be straight-chain, branched or else cyclic. Very particular preference is given to methyl, ethyl and propyl.

Unless stated otherwise, “%” means “% by weight”.

I may be used as the crude product; contents of >40% are sufficient, preference being given to contents of >50%.

II is a complex borohydride such as calcium borohydride or zinc borohydride, preferably an alkali metal borohydride such as sodium borohydride or potassium borohydride, more preferably sodium borohydride.

III is preferably a cerium(III) halide or a halide of other trivalent rare earth metals or a mixture of trivalent rare earth metals, more preferably cerium(III) chloride with a water content of <1% or a water content between 1% and 40%, especially cerium(III) chloride heptahydrate.

Based on compound I, the amount of rare earth compound needed is less than 100 mol %, especially less than 50 mol %, preferably less than 30 mol %, more preferably less than 15 mol %.

V consists preferably of an alkanoic anhydride or alkanoyl halide or alkanoic acid and a water-removing reagent such as propanephosphonic anhydride, isobutyl chloroformate or pivaloyl chloride, more preferably acetyl chloride or acetic anhydride, in conjunction with a base, preferably triethylamine or pyridine. The optionally added catalyst is preferably a hypernucleophilic acylation catalyst, more preferably 4-dimethylaminopyridine.

The reduction is appropriately performed in a solvent or a solvent mixture comprising methanol and optionally water, tetrahydrofuran, 2-methyltetrahydrofuran, tert-butyl methyl ether, diisopropyl ether, dipropyl ether, dibutyl ether, 1,4-dioxane, toluene, xylene, hexane, heptane or petroleum ether. Particular preference is given to performing the reduction in methanol or mixtures of methanol and an ether, this ether more preferably originating from the group of {tetra-hydrofuran, 2-methyltetrahydrofuran, 1,4-dioxane}. In the case of mixtures, preference is given to those with less than 50% methanol, particular preference to those with less than 25%.

The optional acylation to simplify the workup is appropriately performed either in a mixture of methanol and an ether, preferably originating from the group of {tetrahydrofuran, 2-methyltetrahydrofuran, 1,4-dioxane, tert-butyl methyl ether, diisopropyl ether} or an aprotic solvent, preferably originating from the group of {alkanes, arenes, esters, ketones, ethers, N,N-dimethylformamide, dimethyl sulfoxide, N-methyl-pyrrolidone}, or a mixture of methanol with a plurality of such solvents. Particularly preferred solvents or solvent mixtures comprise, as well as methanol, tetrahydrofuran, 2-methyltetrahydrofuran, 1,4-dioxane, tert-butyl methyl ether, ethyl acetate, butyl acetate, isobutyl methyl ketone, toluene, xylene.

The reduction is performed between −100° C. and +50° C., preferably between −100° C. and 0° C., more preferably at −80 to −50° C. The reaction within the preferred and particularly preferred temperature ranges leads to products with relatively high stereoselectivity.

The acylation is performed preferably between −20° C. and room temperature, most preferably at −10 to +10° C.

The reduction and the acylation can be performed in the same solvent. In a preferred embodiment, the reduction is performed in methanol, which is then distilled off and replaced by a higher-boiling aprotic solvent in which the acylation is performed (solvent exchange).

In a further preferred embodiment, the reduction is performed in a mixture of methanol and an aprotic solvent (see above).

The acylation is appropriately performed in the same solvent mixture, preference being given to using an excess of acylating agent in order to convert the methanol content of the solvent to the corresponding methyl ester.

It is also already possible to remove precipitated rare earth metal salts by filtration before the acylation. When this is not possible, the amount of the acylating agent should be increased such that residual water of crystallization is consumed by acylating agent.

The reaction can be performed in such a way that the rare earth metal compound is initially charged together with the starting material I in a solvent or solvent mixture (according to the above definition) and is then admixed in portions with the borohydride II. The preferred metering rate or portion size is selected such that a minimum level of overreduction (1,4 reduction) of the substrate occurs.

A preferred mode of performance of the reduction consists, however, in metering the starting material (optionally in a solvent or solvent mixture according to the above definition) and the borohydride (optionally in a solvent or solvent mixture according to the above definition) in portions and alternately into the initially charged solution or suspension of the rare earth metal compound (in solvent or solvent mixture according to the above definition), the particular reactant preferably being metered in when the amount of the same reactant added beforehand in each case has for the most part been consumed. The size of the individual portions of the starting material is preferably less than the amount of rare earth metal compound used.

A further preferred mode of performance of the reduction consists in reacting a solution or suspension of substrate and rare earth metal compound (optionally in a solvent or solvent mixture as defined above) continuously with the borohydride (optionally in a solvent or solvent mixture as defined above).

The yields in this reaction of (cis)-IV and (cis)-VI, based on the amount of I used, are typically >30%, preferably >50% and more preferably >70%. According to the invention, the ratio of (cis)-IV or (cis)-VI to (trans)-VI or (trans)-VI is better than 7:1, preferably better than 10:1, more preferably better than 15:1, a suitable reaction regime (temperature in the reduction between −50° C. and −100° C.) typically achieving a ratio of (cis)-IV or (cis)-VI to (trans)-VI or (trans)-VI of approx. 20:1. The portion of the by-products which result from “overreduction” (1,4 reduction followed by 1,2 reduction) is not higher than 20 mol % (based on compound I) when the reduction is performed between 0° C. and room temperature, and not more than 10% in the case of performance between −100° C. and −50° C.

The process described offers, for the first time, an industrially and economically realizable preparation route for derivatives of cyclopent-4-ene-1,3-diol, and for the first time demonstrates the performability of a Luche reduction with substoichiometric amounts of rare earth metal compound. It will be illustrated by the examples which follow:

### EXAMPLES

### Example 1

### Operation in Methanol/THF with Alternating Addition at 0° C.

4.2 g of cerium(III) chloride heptahydrate (11.2 mmol) are dissolved in 6.3 g of methanol at room temperature. Then 27.2 g of tetrahydrofuran are added and the mixture is cooled to 0 to 5° C. Then the mixture is admixed at this temperature with exactly one tenth of a solution of 5.0 g of 4-hydroxycyclopent-2-enone (approx. 50% pure) in 5.0 g of THF, and stirred for a further 10 min. Again, exactly one tenth of 0.64 g of sodium borohydride (17.0 mmol) is then added all at once to this mixture at 0 to 5° C. and the mixture is stirred for a further 20 min. Subsequently, the next tenth of reactant solution is added, the mixture is stirred for 10 min, the next tenth of sodium borohydride is added and the mixture is stirred for a further 20 min. This cycle is repeated until all of the reactant solution and all of the sodium borohydride has been added. The mixture is then stirred for another 30 min and then admixed with 41.3 g of triethylamine. 0.25 g of 4-dimethylaminopyridine (2.1 mmol) is then added at 0 to 5° C. as a catalyst to the solution/suspension which forms, and then the mixture is admixed at 0 to 5° C. with 41.6 g of acetic anhydride (407.7 mmol) within approx. 30 min and the mixture is stirred at this temperature for another 120 min in order to complete the conversion. Subsequently, the mixture is admixed first with 42.0 ml of methyl t-butyl ether and then with 42.00 ml of water. The phases are then separated and the organic phase is concentrated by rotary evaporation to as great an extent as possible. After distillation at 0 to 2 mbar using a microstill at to 90° C.@1 mbar, 3.9 g of product are thus obtained, which, according to GC analysis, contains approx. 80% of the desired product, 8% of the transisomer and a total of 10% cis/trans-1,3-diacetoxycyclopentane (VII) (yield: 42% neglecting the purities of reactant and product, and approx. 66% after correcting by the reactant content and product content).

### Example 2

### Operation in Methanol/THF with Alternating Addition at −65° C.

As example 1, except alternating addition of borohydride and reactant solution at −60 to −70° C. The yield of crude product in this operation is virtually unchanged (4.0 g), but the selectivity of the reaction is significantly increased and hence the purity of the product is significantly better. This is true both of the ratio of cis- to trans-cyclopent-4-ene-1,3-diacetate (20:1) and for the proportion of over-reduced compounds in the product (cis/trans-1,3-diacetoxycyclopentane, 2%).

### Example 3

### Operation in Methanol at 0° C., Sodium Borohydride Addition

8.36 g of cerium(III) chloride heptahydrate are dissolved in 25.1 g of methanol, and then 10.0 g of 4-hydroxycyclopent-2-enone (approx. 50% pure) are added. Subsequently, the mixture is cooled to 0° C. and admixed with one tenth of a total of 1.54 g of sodium borohydride (40.8 mmol), and stirred for approx. 30 min. Then it is admixed with a further tenth of sodium borohydride, stirred again for 30 min, and this procedure is continued until all of the borohydride has been added. Subsequently, the mixture is stirred at RT overnight and admixed the next day with 3.9 g of acetone (67.3 mmol) in order to destroy excess sodium borohydride. The resulting mixture is freed of the solvent on a rotary evaporator to as great an extent as possible, admixed with 50.0 g of THF and concentrated by rotary evaporation once again in order to remove as much methanol as possible. Subsequently, the mixture is admixed again with 50.0 g of THF and then with 30.9 g of triethylamine and 0.5 g of 4-dimethylaminopyridine (4.1 mmol) as a catalyst. 31.2 g of acetic anhydride (305.8 mmol) are then added dropwise at 0 to 10° C. to this mixture which is stirred for a further 60 min in order to complete the conversion. Then it is admixed with 50 ml of methyl tert-butyl ether and slowly with 31.0 g of water. After phase separation, the organic phase is freed of the solvent on a rotary evaporator to as great an extent as possible, and the resulting oily residue is distilled using a microstill. 8.4 g of an approx. 75% product are obtained, which, as well as the desired cis-cyclopent-4-ene-1,3-diacetate, also contains approx. 8% cis-cyclopent-4-ene-1,3-diacetate and 15% overreduced impurities. Yield: 44.7 neglecting the reactant and product contents, and approx. 66% after correction by the reactant content and product content.

### Example 4

### Operation in Methanol at −65° C., Sodium Borohydride Addition

As example 3, except addition of borohydride at −60 to −70° C. The yield of crude product in this operation is again virtually unchanged (8.2 g), but the selectivity of the reaction is significantly higher and hence the purity of the product is improved. This applies both to the ratio of cis- to trans-cyclopent-4-ene-1,3-diacetate (20:1) and to the proportion of over-reduced compounds in the product (cis/trans-1,3-diacetoxycyclopentane, 5%).

### Example 5

### Operation in Methanol at −65° C., Alternating Addition

8.36 g of cerium(III) chloride heptahydrate (0.22 eq.) are dissolved in 25.1 g of methanol and cooled to −65° C. Exactly one tenth of a mixture of 10.0 g of 4-hydroxycyclopent-2-enone (approx. 50% pure) and 5.0 g of methanol are then added to this mixture which is stirred for a further 10 min. Then it is admixed at this temperature with exactly one tenth of a total of 1.54 g of sodium borohydride (40.8 mmol) and stirred for a further approx. 20 min. The addition of reactant solution and sodium borohydride is continued in the same way until everything has been added. Subsequently, the mixture is stirred at −65° C. overnight, and admixed the next day with 3.9 g of acetone (67.3 mmol), in order to destroy excess sodium borohydride. The resulting mixture is freed of the solvent on a rotary evaporator to as great an extent as possible, admixed with 50.0 g of THF and concentrated once again by rotary evaporation in order to remove as much methanol as possible. Subsequently, the mixture is admixed again with 50.0 g of THF and then with 30.9 g of triethylamine and 0.5 g of 4-dimethylaminopyridine (4.1 mmol) as a catalyst. 31.2 g of acetic anhydride (305.8 mmol) are then added dropwise to this mixture at 0 to 10° C., and the mixture is stirred for a further 60 min in order to complete the conversion. Then it is admixed with 50 ml of methyl tert-butyl ether and slowly with 31.0 g of water. After phase separation, the organic phase is freed of the solvent on a rotary evaporator to as great an extent as possible, and the resulting oily residue is distilled by means of a microstill. 8.6 g of an approx. 90% pure product are obtained, which, as well as the desired cis-cyclopent-4-ene-1,3-diacetate, also contains approx. 4% cis-cyclopent-4-ene-1,3-diacetate and 2% overreduced impurities. Yield: 45.8% based on reactant tel quel and approx. 82.4% after correction by reactant content (approx. 50% pure) and product content.

1. A process for preparing cis-cyclopent-4-ene-1,3-diol from 4-hydroxycyclopent-2-enone comprising selectively reducing 4-hydroxycyclopent-2-enone with complex borohydrides in the presence of trivalent rare earth metal compounds according to the following equation 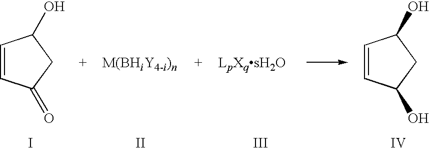
where
M is an alkali metal, an alkaline earth metal, zinc or zirconium,
i is an integer from 1 to 4,
n is an integer from 1 to 4 corresponding to the valency of M,
Y is a monovalent anion,
L is a trivalent rare earth metal or mixtures of trivalent rare earth metals,
X is an anion of an organic or inorganic acid,
p and q are each independently integers from 1 to 4, according to the valency of X and the rare earth metal,
s is any number from 0 to 20 and represents the water content of the rare earth metal salt, and
wherein the rare earth metal compound is in a substoichiometric amount based on the 4-hydroxycyclopent-2-enone.

2. A process for preparing cis-cyclopent-4-ene-1,3-diol dialkanoates from 4-hydroxycyclopent-2-enone as claimed in claim 1 comprising selectively reducing 4-hydroxycyclopent-2-enone with complex borohydrides in the presence of trivalent rare earth metal compounds followed by an acetylation with or without isolation of the cis-cyclopent-4-ene-1,3-diol formed as an intermediate according to the following equation: 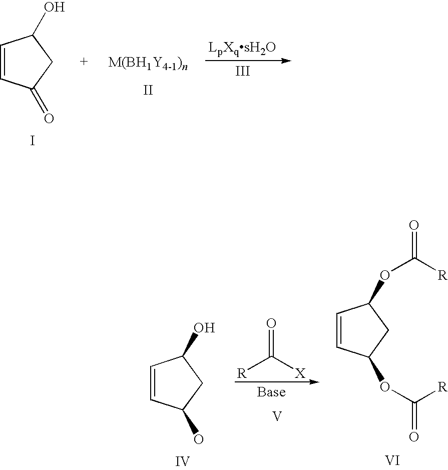
where M, i, Y, n, L, X, p, q and s are each as defined in claim 1 and
R is an aromatic or aliphatic radical and
wherein the rare earth metal compound is in a substoichiometric amount based on the 4-hydroxycyclopent-2-enone.

3. The process as claimed in claim 1, wherein less than 50 mol % of rare earth metal compound or mixed rare earth metal compound, based on 4-hydroxycyclopent-2-enone, is used.

4. The process as claimed in claim 1, wherein the trivalent rare earth metal is cerium.

5. The process as claimed in claim 1, wherein the trivalent rare earth metal salt is hydrated cerium (III) chloride with a water content between 1% and 40%.

6. The process as claimed in claim 1, wherein the trivalent rare earth metal salt is dry cerium (III) chloride with a water content of <1%.

7. The process as claimed in claim 1, wherein the complex borohydride is sodium borohydride, potassium borohydride, calcium borohydride or zinc borohydride.

8. The process as claimed in claim 1, wherein the reduction step is performed in a solvent or solvent mixture comprising methanol and optionally water, tetrahydrofuran, 2 methyltetrahydrofuran, tert-butyl methyl ether, diisopropyl ether, dipropyl ether, dibutyl ether, 1,4-dioxane, toluene, xylene, hexane, heptane or petroleum ether.

9. The process as claimed in claim 2, wherein the acylation step is performed in the same solvent or solvent mixture as the reduction.

10. The process as claimed in claim 2, wherein a solvent exchange takes place between reduction and acylation, and the acylation is performed in an aprotic solvent.

11. The process as claimed in claim 2, wherein the acylation takes place in acetic anhydride or acetyl chloride in the presence of a base.

12. The process as claimed in claim 2, wherein the acetylation comprises an acylating agent obtained in situ by activating a carboxylic acid with the aid of a water-removing activating agent.

13. The process as claimed in claim 2, wherein the process further comprises hydrolyzing cis-cyclopent-4-ene-1,3-diol dialkanoates in an enzyme-mediated reaction to give a nonracemic monoester.

14. The process as claimed in claim 1, wherein the wherein said process further comprises converting said cis-cyclopent-4-ene-1,3-diol in an enzyme-mediated reaction to (1R,4S)-cis-4-acetoxy-2-cyclopenten-1-ol or to (1S,4R)-cis-4-acetoxy-2-cyclopenten-1-ol.
